(19) United States
(12) Patent Application Publication
(10) Pub. No.: US 2026/0240758 A1
(43) Pub. Date: Aug. 20, 2026

(54) INJECTABLE PHENYLEPHRINE COMPOSITIONS

(71) Applicant: Hikma Pharmaceuticals USA Inc., Berkeley Heights, NJ (US)

(72) Inventor: Matija Simac, Zagreb (HR)

(73) Assignee: Hikma Pharmaceuticals USA Inc., Berkeley Heights, NJ (US)

(21) Appl. No.: 18/992,413

(22) PCT Filed: Jul. 6, 2023
(86) PCT No.: PCT/EP2023/068706

§ 371 Date: Jan. 8, 2025

Related U.S. Application Data
(60) Provisional application No. 63/488,325, filed on Mar. 3, 2023.

(30) Foreign Application Priority Data
Jul. 7, 2022 (WO) PCT/EP2022/068891

(51) Int. Cl. A61K 9/00; A61J 1/10; A61K 9/08; A61K 31/137; A61K 47/02; A61K 47/12

(52) U.S. Cl. CPC A61K 9/0019; A61J 1/10; A61K 9/08; A61K 31/137; A61K 47/02; A61K 47/12

(57) ABSTRACT

Described herein is a product including a ready-to-administer injectable formulation, the formulation including phenylephrine or a pharmaceutically acceptable salt thereof, a tonicity agent and a pH adjusting agent, wherein the pH adjusting agent is tartaric acid; and wherein the formulation of this oxygen sensitive substance is stable for prolonged period of times even when manufactured without protection from oxygen and when packed and stored in a flexible plastic container without modification of atmosphere by oxygen removal from the bag or the overwrap.

### FIELD OF THE INVENTION

The present disclosure relates to a ready-to-administer formulation comprising phenylephrine that is stable in a flexible plastic container. Such compositions provide good stability of the phenylephrine over time.

### BACKGROUND

Phenylephrine is an alpha-1 adrenergic receptor agonist indicated for increasing blood pressure in adults with clinically important hypotension resulting primarily from vasodilation, in such settings as septic shock or anesthesia.

The structure of phenylephrine is given below.

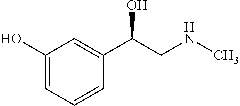

Phenylephrine is sensitive to oxidation and will normally degrade into several oxidation related impurities.

A pathway for phenylephrine oxidative degradation suggested by Rezk et al.; Journal of AOAC International, Vol. 100, No. 2, pp. 434-444, 2017 is shown in Scheme 1.

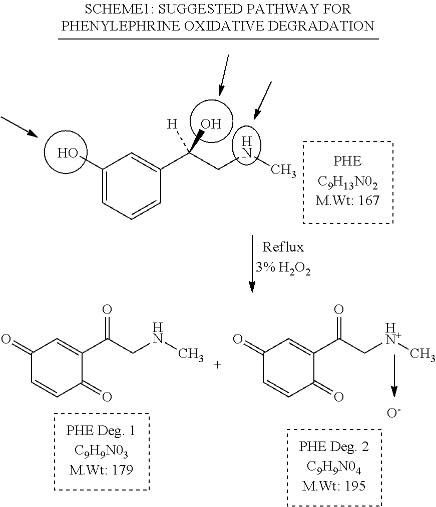

Phenylephrine products that are available in the market are normally stored in glass vials, glass ampules or pre-filled syringes to protect phenylephrine from oxygen and consequently from oxidation. Often these products need to be diluted prior to being administered to the patient.

Jansen et al, Hosp Pharm; 49 (5): 455-457, 2014 has studied the stability of phenylephrine compositions with a phenylephrine HCl concentration of 400 μg/ml and 200 μg/ml stored in polyvinyl chloride (PVC) bags. As seen in the results, the phenylephrine has degraded by around 4-5% after 60 days at room temperature.

US 2021/0228507 A1 mentions phenylephrine compositions and it also mentions that formulations with low concentrations of phenylephrine are less stable than formulations with higher concentrations of phenylephrine.

### SUMMARY

It has been found that a ready-to-administer injectable formulation comprising phenylephrine or a pharmaceutically acceptable salt thereof, an osmolality adjusting agent, and tartaric acid in low amounts, and manufactured without any protection from oxygen possesses surprisingly enhanced stability when stored in a flexible plastic container. Described herein are the formulations and products comprising the formulations contained in a flexible plastic container.

The inventors have also found that the color and stability of the products described herein can be further improved by overwrapping the flexible plastic container with an overwrap. It has been found that when overwrapping the product, the product need not be overwrapped and sealed in or with inert atmosphere. Also, the product does not require any atmosphere controlling methods for reduction of oxygen content in between the product and the overwrap.

The inventors have also found that the ready-to-administer injectable formulation filled in a flexible plastic container as mentioned above could be further protected to prevent water loss by overwrapping the flexible plastic container with an overwrap. Such protection, however, is not necessary.

Because of their enhanced storage stability, the products and formulations disclosed herein can be advantageously stored for relatively long periods. The ready-to-administer injectable formulations mentioned above have an excellent safety profile when administered in high volumes to patients, due to the exceptionally low amounts and to the type of excipients in the formulations.

### DETAILED DISCLOSURE (INCLUDING DEFINITIONS)

The disclosure relates to ready-to-administer injectable formulations of phenylephrine that can further be contained or stored in a flexible plastic container. A product for injection includes an injectable formulation contained or stored in a flexible plastic container.

The ready-to-administer phenylephrine formulations comprise an aqueous solvent.

By the term “aqueous solvent” is understood any composition/solvent in which water is present in or above 50% v/v, such as, e.g., a composition comprising from 50% v/v to 99.5% v/v water, from 50% v/v to 90% v/v water, from 60% v/v to 85% v/v water, from 70% v/v to 80% v/v water. Accordingly, aqueous compositions include compositions comprising 50% v/v or more water, 60% v/v or more water, 70% v/v or more water, 75% v/v or more water, 80% v/v or more water, 85% v/v or more water, 90% v/v or more water, 95% v/v or more water, or 99% v/v or more water. The aqueous composition can additionally comprise pharmaceutically acceptable organic solvents. The aqueous composition may further comprise standard diluents for parenteral use, such as water for injection, 0.9% sodium chloride for injection, dextrose 5% for injection or other suitable diluents.

All of the numbers used herein, except for pH, are modified by the term “about.” This means that each number includes minor variations as defined ±10% of the numerical value or range in question.

The term “phenylephrine” as used herein means phenylephrine or a pharmaceutically acceptable salt of phenylephrine.

In an aspect, phenylephrine is phenylephrine HCl.

When specific amounts or ranges of amounts of phenylephrine are given in this application, all values are calculated based on phenylephrine HCl.

In an aspect, the concentration of phenylephrine in the ready-to-administer formulation is from 0.05 to 0.5 mg/ml.

In an aspect, the concentration of phenylephrine may be 0.05 mg/ml, 0.06 mg/ml, 0.07 mg/ml, 0.08 mg/ml, 0.09 mg/ml, 0.10 mg/ml, 0.11 mg/ml, 0.12 mg/ml, 0.13 mg/ml, 0.14 mg/ml, 0.15 mg/ml, 0.16 mg/ml, 0.17 mg/ml, 0.18 mg/ml, 0.19 mg/ml, 0.20 mg/ml, 0.21 mg/ml, 0.22 mg/ml, 0.23 mg/ml, 0.24 mg/ml, 0.25 mg/ml, 0.26 mg/ml, 0.27 mg/ml, 0.28 mg/ml, 0.29 mg/ml, 0.30 mg/ml, 0.31 mg/ml, 0.32 mg/ml, 0.33 mg/ml, 0.34 mg/ml, 0.35 mg/ml, 0.36 mg/ml, 0.37 mg/ml, 0.38 mg/ml, 0.39 mg/ml, 0.40 mg/ml, 0.41 mg/ml, 0.42 mg/ml, 0.43 mg/ml, 0.44 mg/ml, 0.45 mg/ml, 0.46 mg/ml, 0.47 mg/ml, 0.48 mg/ml, 0.49 mg/ml or 0.50 mg/ml.

In an aspect, the concentration of phenylephrine in the ready-to-administer formulation is 0.08 mg/ml.

In an aspect, the concentration of phenylephrine in the ready-to-administer formulation is 0.16 mg/ml.

In an aspect, the concentration of phenylephrine in the ready-to-administer formulation is 0.20 mg/ml.

In an aspect, the concentration of phenylephrine in the ready-to-administer formulation is 0.40 mg/ml.

The pH of the formulations is in the range from 3.5 to 4.5. In another aspect, the pH is in the range of 3.7-4.4. In another aspect the pH is in the range of 3.8-4.3. In another aspect the pH is in the range of 3.8-4.2, 3.9-4.2 or 3.9-4.1. In yet another aspect the pH is 4.0.

In an aspect, the pH of the formulations may be 3.5, 3.6, 3.7, 3.8, 3.9, 4.0, 4.1, 4.2, 4.3, 4.4 or 4.5.

“pH” is the conventional measurement unit of hydrogen ion activity in a solution at room temperature unless another temperature is specified.

As used herein, the term “pH” in the compositions is defined as +0.1 of the numerical value or range in question.

In an aspect, pH values are given for the formulations just after preparation, which means at the start of the shelf life.

In an aspect, pH values are given for the formulations after 3 months of storage at 40° C.

In one aspect, pH values are given for the formulations after 6 months of storage at 40° C.

As used herein, the months of storage are calculated from the time of preparation of the ready-to-administer formulation.

In another aspect, pH values are given for the formulation after certain period of time in predetermined temperature conditions.

In one aspect, the composition is an isosmotic composition. It is to be understood that the term “isosmotic” in accordance with the present disclosure means having similar osmolality to the physiologic osmolality of blood.

Typically, the ready-to-administer pharmaceutical compositions have osmolality from 240 to 340 mOsm/kg.

In one aspect, the composition has osmolality from 240 to 600 mOsm/kg.

In one aspect, the ready-to-administer formulation included in the ready-to-administer product is both isotonic and has an osmolality similar to the physiological osmolality of blood as described above.

In some aspects, the ready-to-administer compositions further comprise one or more osmolality adjusting agents. Suitable osmolality adjusting agents for use in ready-to-administer compositions include but are not limited to sodium chloride and dextrose.

In an aspect, sodium chloride is used as an osmolality adjusting agent.

In an aspect, the concentration of sodium chloride is in the range of 8 to 10 mg/ml.

In an aspect, the concentration of sodium chloride may be 8 mg/ml, 8.5 mg/ml, 9 mg/ml, 9.5 mg/ml or 10 mg/ml.

In an aspect, the concentration of sodium chloride is 9 mg/ml.

In an aspect, the formulation comprises a low amount of a pH adjusting agent.

The term “low amount” used herein means a concentration of the pH adjusting agent is in the range of 0.001 mg/ml to 0.05 mg/ml, more preferably in the range of 0.005 mg/ml to 0.05 mg/ml.

In an aspect, the concentration of the pH adjusting agent should be in range to adjust the pH of the formulation in the specific range.

In an aspect, the pH in the formulations should be adjusted to a pH within the range from 3.5 to 4.5.

In an aspect, pH adjusting agent is tartaric acid.

In an aspect, the concentration of tartaric acid in the formulation is in the range of 0.001 mg/ml to 0.05 mg/ml

In an aspect, the concentration of tartaric acid in the formulation is in the range of 0.001 mg/ml to 0.04 mg/ml.

In an aspect, the concentration of tartaric acid in the formulation is in the range of 0.001 mg/ml to 0.035 mg/ml.

In an aspect, the concentration of tartaric acid in the formulation is in the range of 0.001 mg/ml to 0.03 mg/ml.

In an aspect, the concentration of tartaric acid in the formulation is in the range of 0.001 mg/ml to 0.025 mg/ml.

In an aspect, the concentration of tartaric acid in the formulation is in the range of 0.001 mg/ml to 0.023 mg/ml.

In an aspect, the concentration of tartaric acid in the formulation is in the range of 0.001 mg/ml to 0.022 mg/ml.

In an aspect, the concentration of tartaric acid in the formulation is in the range of 0.001 mg/ml to 0.020 mg/ml.

In an aspect, a ready-to-administer injectable product comprises a formulation of phenylephrine or a pharmaceutically acceptable salt thereof, a tonicity agent, an aqueous solvent, and a low amount of the pH adjusting agent tartaric acid, contained in a flexible plastic container.

In an aspect, a ready-to-administer injectable product comprises a formulation of phenylephrine or a pharmaceutically acceptable salt thereof, a tonicity agent, an aqueous solvent, and the pH adjusting agent tartaric acid in an amount less than 0.05 mg/ml, contained in a flexible plastic container.

In an aspect, a ready-to-administer injectable product comprises a formulation of phenylephrine or a pharmaceutically acceptable salt thereof, a tonicity agent, an aqueous solvent, and the pH adjusting agent tartaric acid in an amount from 0.001 mg/ml to 0.05 mg/ml, contained in a flexible plastic container.

In an aspect, a ready-to-administer injectable product that requires no removal of oxygen from the product, comprises a formulation of phenylephrine or a pharmaceutically acceptable salt thereof, an osmolality adjusting agent, an aqueous solvent, and a low amount of the pH adjusting agent tartaric acid contained in a flexible plastic container. As used herein, removal of oxygen may include purging the headspace of the flexible plastic container with an inert gas and, or inert gas sparging of the formulation with an inert gas, and hermetically sealing of the container and/or packing the flexible plastic container containing the solution with an oxygen absorber into another container (e.g. aluminum pouch). In other words, no measures are taken to reduce the amount of oxygen in the formulation or in the headspace of the container.

In an aspect, a ready-to-administer injectable product that requires no removal of oxygen from the product, comprises a formulation of phenylephrine or a pharmaceutically acceptable salt thereof, an osmolality adjusting agent, an aqueous solvent, and the pH adjusting agent tartaric acid in an amount less than 0.05 mg/ml, contained in a flexible plastic container.

In an aspect, a ready-to-administer injectable product that requires no removal of oxygen from the product, comprises a formulation of phenylephrine or a pharmaceutically acceptable salt thereof, an osmolality adjusting agent, an aqueous solvent and the pH adjusting agent tartaric acid in an amount less than 0.05 mg/ml, contained in a flexible plastic container and wherein the level of total impurities is less than 0.4% after 3 months at 40 degrees Celsius as determined by HPLC.

In an aspect, a ready-to-administer injectable product that requires no removal of oxygen from the product, comprises a formulation of phenylephrine or a pharmaceutically acceptable salt thereof, an osmolality adjusting agent, an aqueous solvent, and the pH adjusting agent tartaric acid in an amount from 0.001 mg/ml to 0.05 mg/ml, contained in a flexible plastic container.

In an aspect, a ready-to-administer injectable product that requires no removal of oxygen from the product, comprises a formulation of phenylephrine or a pharmaceutically acceptable salt thereof, an osmolality adjusting agent, an aqueous solvent, and the pH adjusting agent tartaric acid in an amount from 0.001 mg/ml to 0.05 mg/ml, contained in a flexible plastic container and wherein the level of total impurities is less than 0.4% after 3 months at 40 degrees Celsius as determined by HPLC.

In an aspect, a ready-to-administer injectable product that requires no removal of oxygen from the product, consists essentially of a formulation of phenylephrine or a pharmaceutically acceptable salt thereof, an osmolality adjusting agent, an aqueous solvent, and a low amount of the pH adjusting agent tartaric acid contained in a flexible plastic container.

In an aspect, a ready-to-administer injectable product that requires no removal of oxygen from the product, consists essentially of a formulation of phenylephrine or a pharmaceutically acceptable salt thereof, an osmolality adjusting agent, an aqueous solvent, and the pH adjusting agent tartaric acid in an amount less than 0.05 mg/ml, contained in a flexible plastic container.

In an aspect, a ready-to-administer injectable product that requires no removal of oxygen from the product, consists essentially of a formulation of phenylephrine or a pharmaceutically acceptable salt thereof, an osmolality adjusting agent, an aqueous solvent, and the pH adjusting agent tartaric acid in an amount less than 0.05 mg/ml, contained in a flexible plastic container and wherein the level of total impurities is less than 0.4% after 3 months at 40 degrees Celsius as determined by HPLC.

In an aspect, a ready-to-administer injectable product that requires no removal of oxygen from the product, consists essentially of a formulation of phenylephrine or a pharmaceutically acceptable salt thereof, an osmolality adjusting agent, an aqueous solvent, and the pH adjusting agent tartaric acid in an amount from 0.001 mg/ml to 0.05 mg/ml, contained in a flexible plastic container.

In an aspect, a ready-to-administer injectable product that requires no removal of oxygen from the product, consists essentially of a formulation of phenylephrine or a pharmaceutically acceptable salt thereof, an osmolality adjusting agent, an aqueous solvent, and the pH adjusting agent tartaric acid in an amount from 0.001 mg/ml to 0.05 mg/ml, contained in a flexible plastic container and wherein the level of total impurities is less than 0.4% after 3 months at 40 degrees Celsius as determined by HPLC.

In an aspect, a ready-to-administer injectable product consists of a formulation of phenylephrine or a pharmaceutically acceptable salt thereof, an osmolality adjusting agent, an aqueous solvent, and a low amount of the pH adjusting agent tartaric acid, contained in a flexible plastic container.

In an aspect, a ready-to-administer injectable product consists of a formulation of phenylephrine or a pharmaceutically acceptable salt thereof, an osmolality adjusting agent, an aqueous solvent, and the pH adjusting agent tartaric acid in an amount less than 0.05 mg/ml, contained in a flexible plastic container.

In an aspect, a ready-to-administer injectable product consists of a formulation of phenylephrine or a pharmaceutically acceptable salt thereof, an osmolality adjusting agent, an aqueous solvent, and the pH adjusting agent tartaric acid in an amount from 0.001 mg/ml to 0.05 mg/ml, contained in a flexible plastic container.

In an aspect, a ready-to-administer injectable product that requires no removal of oxygen from the product, consists of a formulation of phenylephrine or a pharmaceutically acceptable salt thereof, an osmolality adjusting agent, an aqueous solvent, and a low amount of the pH adjusting agent tartaric acid, contained in a flexible plastic container.

In an aspect, a ready-to-administer injectable product that requires no removal of oxygen from the product, consists of a formulation of phenylephrine or a pharmaceutically acceptable salt thereof, an osmolality adjusting agent, an aqueous solvent, and the pH adjusting agent tartaric acid in an amount less than 0.05 mg/ml, contained in a flexible plastic container.

In an aspect, a ready-to-administer injectable product that requires no removal of oxygen from the product, consists of a formulation of phenylephrine or a pharmaceutically acceptable salt thereof, an osmolality adjusting agent, an aqueous solvent, and the pH adjusting agent tartaric acid in an amount less than 0.05 mg/ml, contained in a flexible plastic container and wherein the level of total impurities is less than 0.4% after 3 months at 40 degrees Celsius as determined by HPLC.

In an aspect, a ready-to-administer injectable product that requires no removal of oxygen from the product, consists of a formulation of phenylephrine or a pharmaceutically acceptable salt thereof, an osmolality adjusting agent, an aqueous solvent, and the pH adjusting agent tartaric acid in an amount from 0.001 mg/ml to 0.05 mg/ml, contained in a flexible plastic container.

In an aspect, a ready-to-administer injectable product that requires no removal of oxygen from the product, consists of a formulation of phenylephrine or a pharmaceutically acceptable salt thereof, an osmolality adjusting agent, an aqueous solvent, and the pH adjusting agent tartaric acid in an amount from 0.001 mg/ml to 0.05 mg/ml, contained in a flexible plastic container and wherein the level of total impurities is less than 0.4% after 3 months at 40 degrees Celsius as determined by HPLC.

In an aspect, a ready-to-administer injectable product consists of phenylephrine or a pharmaceutically acceptable salt thereof, sodium chloride, low amount of tartaric acid, and an aqueous solvent, contained in a flexible plastic container.

In an aspect, a ready-to-administer injectable product consists of phenylephrine or a pharmaceutically acceptable salt thereof, sodium chloride, the amount of tartaric acid is less than 0.05 mg/ml, and an aqueous solvent, contained in a flexible plastic container.

In an aspect, a ready-to-administer injectable product consists of phenylephrine or a pharmaceutically acceptable salt thereof, sodium chloride, the amount of tartaric acid is from 0.001 mg/ml to 0.05 mg/ml, and an aqueous solvent, contained in a flexible plastic container.

In an aspect, a ready-to-administer injectable product that requires no removal of oxygen from the product, consists of phenylephrine or a pharmaceutically acceptable salt thereof, sodium chloride, low amount of tartaric acid, and an aqueous solvent, contained in a flexible plastic container.

In an aspect, a ready-to-administer injectable product that requires no removal of oxygen from the product, consists of phenylephrine or a pharmaceutically acceptable salt thereof, sodium chloride, the amount of tartaric acid is less than 0.05 mg/ml, and an aqueous solvent, contained in a flexible plastic container.

In an aspect, a ready-to-administer injectable product that requires no removal of oxygen from the product, consists of phenylephrine or a pharmaceutically acceptable salt thereof, sodium chloride, the amount of tartaric acid is from 0.001 mg/ml to 0.05 mg/ml, and an aqueous solvent, contained in a flexible plastic container.

In an aspect, the ready-to-administer formulation does not comprise citric acid.

In an aspect, the ready-to-administer formulation does not comprise citrate.

In an aspect, the ready-to-administer formulation does not comprise an acetate buffer.

In an aspect, the ready-to-administer formulation does not comprise sodium metabisulfite.

In an aspect, the ready-to-administer formulation does not comprise edetate disodium (EDTA).

Ready-to-administer means a formulation that does not need any dilution prior to administration to the patient. A ready-to-administer composition is synonymous with ready-to-infuse or ready-to-inject and it is suitable to be administered directly to the patient.

As used herein, the terms “pharmaceutical composition”, “pharmaceutical formulation”, “composition” and “formulation” are used interchangeably.

In an aspect, phenylephrine in a ready-to-administer product and/or formulation according to the present disclosure, is stable at a temperature of from 2° C. to 8° C. for a certain period of time.

In an aspect, phenylephrine in a ready-to-administer product and/or formulation according to the present disclosure, is stable under room temperature conditions for a certain period of time. The term “room temperature” used herein, is from 20° C. to 27° C.

In an aspect, phenylephrine in a ready-to-administer product and/or formulation according to the present disclosure, is stable at 40° C. for a certain period of time.

In an aspect, phenylephrine in the product and/or formulation described herein is stable over time periods of 7 days (1 week), 14 days (2 weeks), 30 days (1 month), not less than 60 days (2 months), 3 months, 4 months, 180 days (6 months), 9 months, 12 months (1 year), 14 months, 16 months, 18 months, 20 months, 24 months or more at certain specified temperature conditions. The skilled person will acknowledge that a composition comprising an active pharmaceutical ingredient may be stored at refrigerated conditions, intermediate conditions (between refrigerated and room temperature) or at about room temperature conditions. When referring to “certain specified temperature conditions”, it is to be understood to include refrigerated conditions at 2-8° C., intermediate conditions at 10° C. to 18° C. and room temperature conditions at 20° C. to 27° C.

The term “certain period of time” or “relatively long periods” used herein covers the above time periods.

The term “stability”, “chemical stability” or “stable” intends to mean that the product, composition or formulation exhibits an acceptable amount of phenylephrine being present, or not more than a certain amount of phenylephrine has degraded after a certain period of time. Accordingly, in a stable product, solution or formulation unacceptable degradation of API is avoided.

Stability can be presented as the assay of phenylephrine in a product according to the disclosure. If the product, formulation or composition initially contains phenylephrine in a certain assay, the stability of the product, formulation or composition will be reflected by a decrease in the assay of phenylephrine in the product, formulation or composition over time, where a stable product, solution or composition would contain the phenylephrine in a specified assay after a predetermined time period.

In an aspect, the formulation has no more than 10% of assay decrease/drop of phenylephrine in the pharmaceutical formulation, determined by liquid chromatography, e.g., HPLC, UPLC, LC/MS.

For example, a stable composition can be one which has not more than 1%, 2%, 3%, 4%, 5%, 6%, 7%, 8%, 9%, 10%, of assay decrease/drop of phenylephrine after a predetermined time period, determined by liquid chromatography, e.g., HPLC, UPLC, LC/MS.

As used herein, a drop in phenylephrine assay is measured from the time of preparation of the formulation to the specified time, e.g., 3 months and 6 months under specified storage conditions.

When describing stability of a pharmaceutical product, formulation or composition, as little degradation of the active ingredient as possible is of course an important factor and aimed for. However, another important factor is the formation of impurities, which may be formed by degradation of the active ingredient.

In the tables below, one degradation impurity of phenylephrine is identified based on the relative retention time (RRT) in the HPLC chromatogram. This impurity is believed to be a degradation product related to oxidation of phenylephrine. The inventors have found that one of the main oxidative impurities is the impurity RRT 0.66 shown in the examples below.

Accordingly, “stability” may also be defined by the amount of total or specific impurities generated after a certain period of time. The amount of impurities being present may be expressed as a percentage, for example as a peak-area percentage of a HPLC chromatogram or calculated according to standard solution.

As used herein, an increase in total or in specific impurities is measured from the time of preparation of the formulation to the specified time, e.g., 3 months and 6 months under specified storage conditions.

In the following embodiments, when the product includes an overwrap, it is preferred that the overwrap comprises no oxygen absorber or scavenger.

In an aspect, the amount of total impurities in the formulation after 3 months at 40 degrees Celsius, in plastic containers without an overwrap, is less than 0.80% as determined by HPLC.

In an aspect, the amount of total impurities in the formulation after 3 months at 40 degrees Celsius, in plastic containers without an overwrap, is less than 0.30, 0.35, 0.40, 0.45, 0.50, 0.55, 0.60, 0.65, 0.7, 0.75 or 0.80% as determined by HPLC.

In an aspect, the amount of total impurities in the formulation after 3 months at 40 degrees Celsius, in plastic containers without an overwrap, is not more than 0.4% as determined by HPLC.

In an aspect, the amount of total impurities in the formulation after 3 months at 40 degrees Celsius, in plastic containers with an overwrap, is not more than 0.40% as determined by HPLC.

In an aspect, the amount of total impurities in the formulation after 3 months at 40 degrees Celsius, in plastic containers with an overwrap, is not more than 0.05%, 0.10%, 0.15%, 0.20%, 0.25%, 0.30%, 0.35% or 0.40% as determined by HPLC.

In an aspect, the amount of total impurities in the formulation after 3 months at 40 degrees Celsius, in plastic containers with an overwrap, is not more than 0.30% as determined by HPLC.

In an aspect, the amount of total impurities in the formulation after 3 months at 40 degrees Celsius, in plastic containers without an overwrap, in a formulation where the phenylephrine concentration is from 0.05 to 0.5 mg/ml or the concentration is 0.08 mg/ml, 0.16 mg/ml, 0.20 mg/ml or 0.40 mg/ml, is not more than 0.80% as determined by HPLC.

In an aspect, the amount of total impurities in the formulation after 3 months at 40 degrees Celsius, in plastic containers without an overwrap, in a formulation where the phenylephrine concentration is from 0.05 to 0.5 mg/ml or the concentration is 0.08 mg/ml, 0.16 mg/ml, 0.20 mg/ml or 0.40 mg/ml, is less than 0.30, 0.35, 0.40, 0.45, 0.50, 0.55, 0.60, 0.65, 0.7, 0.75 or 0.80% as determined by HPLC.

In an aspect, the amount total impurities in the formulation after 3 months at 40 degrees Celsius, in plastic containers without an overwrap, in a formulation where the phenylephrine concentration is from 0.05 to 0.5 mg/ml or the concentration is 0.08 mg/ml, 0.16 mg/ml, 0.20 mg/ml or 0.40 mg/ml, is not more than 0.40% as determined by HPLC.

In an aspect, the amount of total impurities in the formulation after 3 months at 40 degrees Celsius, in plastic containers with an overwrap, in a formulation where the phenylephrine concentration is from 0.05 to 0.5 mg/ml or the concentration is 0.08 mg/ml, 0.16 mg/ml, 0.20 mg/ml or 0.40 mg/ml, is less than 0.40% as determined by HPLC.

In an aspect, the amount of total impurities in the formulation after 3 months at 40 degrees Celsius, in plastic containers with an overwrap, in a formulation where the phenylephrine concentration is from 0.05 to 0.5 mg/ml or the concentration is 0.08 mg/ml, 0.16 mg/ml, 0.20 mg/ml or 0.40 mg/ml, is less than 0.40%, 0.35%, 0.30%, 0.25%, 0.20%, 0.15% or 0.10% as determined by HPLC.

In an aspect, the amount of total impurities in the formulation after 3 months at 40 degrees Celsius, in plastic containers with an overwrap, in a formulation where the phenylephrine concentration is from 0.05 to 0.5 mg/ml or the concentration is 0.08 mg/ml, 0.16 mg/ml, 0.20 mg/ml or 0.40 mg/ml, is not more than 0.30% as determined by HPLC.

In an aspect, the amount of impurity RRT 0.66 in the formulation after 6 months at 40 degrees Celsius, in plastic containers without an overwrap, is not more than 0.50% as determined by HPLC.

In an aspect, the amount of impurity RRT 0.66 in the formulation after 6 months at 40 degrees Celsius, in plastic containers without an overwrap, is not more than 0.25%, 0.30%, 0.35%, 0.40%, 0.45% or 0.50% as determined by HPLC.

In an aspect, the amount of impurity RRT 0.66 in the formulation after 6 months at 40 degrees Celsius, in plastic containers without an overwrap, in a formulation where the phenylephrine concentration is from 0.15 to 0.25 mg/ml or the concentration is 0.16 mg/ml or 0.20 mg/ml, is not more than 0.50% as determined by HPLC.

In an aspect, the amount of impurity RRT 0.66 in the formulation after 6 months at 40 degrees Celsius, in plastic containers without an overwrap, in a formulation where the phenylephrine concentration is from 0.15 to 0.25 mg/ml or the concentration is 0.16 mg/ml or 0.20 mg/ml, is not more than 0.25%, 0.30%, 0.35%, 0.40%, 0.45% or 0.50% as determined by HPLC.

In addition to chemical stability, the appearance of the formulation may be monitored. Appearance includes visual inspection of precipitation, clarity, and color of the formulation.

Color can also be determined spectrophotometrically by using the L*a*b* color space method and calculating the ΔE in accordance with USP <1061>.

As used herein, the terms “secondary packaging” and “overwrap” are used interchangeably.

To further improve the stability and/or the appearance of the formulations, especially reducing the formation of impurities and formation of color, of the formulations described herein, the flexible plastic containers may be overwrapped with a secondary packaging.

In an aspect, the overwrap comprises a material that provides a barrier to migration of water and/or gases, but does not contain or include components for atmosphere modification within the pouch.

Thus, in an aspect, the flexible plastic container is overwrapped with a secondary packaging. In an aspect, the overwrap is an aluminum pouch in which the flexible plastic container containing the ready-to-administer injectable formulation, is placed. After placement of the container inside the aluminum pouch, the aluminum pouch is sealed. Prior to sealing, there is no exchange/modification of atmosphere or removal of gases needed for assuring stability of formulation.

In an aspect, the flexible container comprises a composition that has not been manufactured in an oxygen-free atmosphere nor manipulated to reduce oxygen level in the product.

In one aspect, the ready-to-administer formulation is contained in a flexible plastic container that is overwrapped with an overwrap, wherein the overwrap comprises no oxygen absorber or scavenger.

In an aspect, the flexible plastic container containing the ready-to-administer formulation is not overwrapped.

As described herein the ready-to-administer phenylephrine formulations are contained in a container. The container may be any container suitable to store a ready-to-administer phenylephrine formulation. Preferably the container is a flexible plastic container. As used herein, the term “flexible plastic container” means flexible polymeric infusion bags or other polymeric containers. Exemplary flexible plastic containers are made of polyolefins, such as polyethylene, polypropylene, copolymers and derivatives thereof, with or without other additives. Examples of marketed films used for manufacture of said plastic containers, e.g., films that could be used are the Nexcel™ M312A, Infuflex 7233 or Polycine APP114-S.

In an aspect, the formulation is contained in a plastic container having multiple layers of different polymers.

In another aspect, the formulation is contained in a plastic container having 3-6 layers of different polymers, wherein the inner layer comprises polypropylene.

In an aspect, the polypropylene may be chemically modified.

In an aspect, the formulation is contained in a plastic container, wherein the inner layer consists of ethylene-propylene copolymer.

In an aspect, the formulation is contained in a plastic container, wherein the inner layer comprises an ethylene-propylene copolymer.

In an aspect, the formulation is contained in a plastic container, wherein the inner layer consists of polypropylene.

In an aspect the polypropylene may be chemically modified.

In an aspect, the formulation is contained in a plastic container, wherein the inner layer comprises a polypropylene.

In an aspect the polypropylene may be chemically modified.

In an aspect, the formulation is contained in a plastic container, where the inner layer consists of polyolefin, and/or a styrene-block copolymer.

In an aspect, the formulation is contained in a plastic container, where the inner layer comprises a polyolefin, and/or a styrene-block copolymer.

In an aspect, the products and formulations in the included aspects are produced under conditions in which dissolved oxygen in the formulation is not removed nor significantly reduced.

The disclosure also provides processes for manufacturing or preparing a product comprising phenylephrine or a pharmaceutically acceptable salt of phenylephrine. The process comprises a) providing water in a tank; b) adding excipients such as an osmolality adjusting agent, a pH adjusting agent, or a combination thereof, to the tank to form a solution; c) adding the phenylephrine or salt thereof to the solution and mixing until the phenylephrine is dissolved; d) measuring the pH and if needed further adjusting the pH to reach a targeted pH of the formulation; e) making up a final batch volume by adding in more water to the solution; f) filtering the solution and filling the solution into flexible plastic containers; g) closing the flexible plastic containers, and h) optionally sterilizing the flexible plastic containers. Sterilization can for example be done by autoclaving.

If a filtration step is applied to the aqueous formulation before filling it into the flexible plastic container, this step may be performed by passing the aqueous formulation through a sterilizing grade filter. Passage may be facilitated by pressurizing the solution with gas.

Optionally, the flexible plastic container comprising the aqueous phenylephrine formulation can be overwrapped with a secondary packaging. The overwrap can for example be a tube wherein the flexible plastic container comprising the aqueous phenylephrine formulation is placed and the tube is sealed in both ends. The overwrap can also be two sheets wherein the bag comprising the aqueous phenylephrine formulation is placed in between the two sheets and the two sheets are then sealed on all four edges.

“Pharmaceutically acceptable” indicates that the substance or composition must be compatible, chemically and/or toxicologically, with the other ingredients comprising a formulation, and/or the mammal being treated therewith.

In an aspect, the formulation in the product is administered parenterally.

In an aspect, the formulation is used to increase blood pressure in adults with clinically important hypotension resulting primarily from vasodilation, in such settings as septic shock or anesthesia.

### EXAMPLES

The preparation of the formulations is described for formulations having a phenylephrine HCl content of 0.2 mg/ml or 0.08 mg/ml, however formulations having other concentrations of phenylephrine, such as for example 0.16 mg/ml or 0.4 mg/ml are prepared in a similar manner.

### Inventive Formulations 1-3

In 900 mL of water, 9 g of sodium chloride was added and mixed until dissolved. The pH was adjusted to a target pH of 4.0 using a 0.06 mol/L solution of tartaric acid.

0.200 g of phenylephrine hydrochloride (Formulation 1 and Formulation 3) or 0.080 g of phenylephrine hydrochloride (Formulation 2) was then added to the solution and mixed until dissolved.

The pH was measured and, if needed, the pH was further adjusted with 0.06 mol/L tartaric acid solution to reach the target pH of 4.0. If needed, more water was added to the solution to make up the volume to a total of 1000 ml.

The solution was filtered through a 0.22 μm filter and filled into flexible plastic bags which were then sealed.

For Formulations 1 and 2, the bags were then autoclaved in an autoclave at 121° C. for 15 min. After autoclaving, the bags were sealed in an aluminum overwrap containing no oxygen absorber or scavenger. For Formulation 3, the bags were sealed in an aluminum overwrap containing no oxygen absorber or scavenger and autoclaved in an autoclave at 121° C. for 15 min.

### Comparative Formulation 4

900 mL of water was purged with nitrogen to a dissolved oxygen level of 2.0 ppm or lower. 9 g of sodium chloride was added and mixed until dissolved. The pH was adjusted to a target pH of 4.0 using a 1 mol/L solution of citric acid.

0.200 g of phenylephrine hydrochloride was then added to the solution and mixed until dissolved under a blanket of nitrogen.

The pH was measured and, if needed, the pH was further adjusted with 1 mol/L citric acid solution to reach the target pH of 4.0. If needed, more water was added to the solution to make up the volume to a total of 1000 ml and solution was overlaid with nitrogen.

The solution was pump filtered through a 0.22 μm filter and filled into flexible plastic bags which were then sealed.

The bags were sealed in an aluminum overwrap containing no oxygen absorber or scavenger and autoclaved in an autoclave at 121° C. for 15 min.

### Analytical Methods

### HPLC Assay Method

Mobile phase A: Phosphoric acid and water (1:1000)
Mobile phase B: Acetonitrile

The gradient of mobile phases is shown below.

| Time (min) | Mobile phase A (%) | Mobile phase B (%) |
|---|---|---|
| 0 | 98 | 2 |
| 2.5 | 98 | 2 |
| 6 | 65 | 35 |
| 6.1 | 98 | 2 |
| 9 | 98 | 2 |

Standard solution: 0.1 mg/mL of phenylephrine hydrochloride RS in water.
Sample solution: Nominally 0.1 mg/mL of phenylephrine hydrochloride

### Chromatographic System

Mode: LC
Detector: UV 273 nm.
Column: 4.6×15 cm; 2.6 μm packing L1
Column temperature: 35° C.
Flow rate: 1.0 mL/min

### Calculation

Calculate the percentage of the labeled amount of phenylephrine hydrochloride.

$$Result = ( \frac{{r}_{u}}{{r}_{s}} ) \times ( \frac{{c}_{s}}{{c}_{u}} ) \times 100$$

ru—peak response of phenylephrine from the Sample solution
rs—peak response of phenylephrine from the Standard solution
cs—concentration of phenylephrine hydrochloride RS in the standard solution (mg/mL)
cu—nominal concentration of phenylephrine hydrochloride RS in the sample solution (mg/mL)

### 1. HPLC Impurities Method

Mobile phase A, Mobile phase b, gradient of mobile phases and sample solution are the same as mentioned above in the HPLC Assay method.

System suitability solution: 0.1 mg/mL of phenylephrine hydrochloride RS and 0.005 mg/mL of phenylephrine related compound F RS in water.

Sensitivity solution: 0.1 μg/mL of phenylephrine HCl RS in water Standard Solution: 0.0002 mg/mL of phenylephrine HCl RS in water

The chromatographic system is the same as mentioned above in the HPLC Assay method except that the detector is UV 215 nm.

In the formulations including tartaric acid, a UV detector of 273 nm was used to calculate the degradation product at RRT 0.66.

### Calculation

Calculate the percentage of each degradation product (see *UV 273 nm above):

$$Result = ( \frac{{r}_{u}}{{r}_{s}} ) \times ( \frac{{c}_{s}}{{c}_{u}} ) \times ( \frac{1}{F} ) \times 100$$

ru—peak response of each degradation product from the sample solution
rs—peak response of phenylephrine from the standard solution
cs—concentration of phenylephrine hydrochloride RS in the standard solution (mg/mL)
cu—nominal concentration of phenylephrine hydrochloride RS in the sample solution (mg/mL)
F—relative response factor, see Table 2.

### 2. Color Spectrophotometric Method (L*a*B* Color Space Method)

The different formulations are measured by UV/VIS spectrometer such as, for example, a Perkin Elmer 650. The different formulations are measured as is without any further preparation.

The total Color Difference ΔE* is

$$Δ {E}^{*} = {[ ( Δ {L}^{*} ) 2 + ( Δ {a}^{*} ) 2 + ( Δ {b}^{*} ) 2 ]}^{1 / 2}$$

in which ΔL*, Δa*, and Δb* are the differences in color coordinates of the specimens being compared.

### Color Coordinates

The Color coordinates, L*, a*, and b* are defined by

$${L}^{*} = 116 {( Y / Y 0 )}^{1 / 3} - 16 ,$$

$${a}^{*} = 5 \; 00 [ {( X / X 0 )}^{1 / 3} - {( Y / Y 0 )}^{1 / 3} ] , and$$

$${b}^{*} = 2 \; 00 [ {( Y / Y 0 )}^{1 / 3} - {( Z / Z 0 )}^{1 / 3} ]$$

in which X0, Y0, and Z0 are the tristimulus values of the nominally white or colorless standard, and Y/Y0 >0.01. Usually they are equal to the tristimulus values of the standard illuminant, with Y0 set equal to 100.0. In this case X0=98.0 and Z0=118.1.

### Example 1

Table 1 shows the effect tartaric acid and phenylephrine concentration on stability compared with citric acid when the formulation is contained in a flexible plastic container with an overwrap.

TABLE 1
STABILITY AT 40° C. WITH OVERWRAP

| | Condition | Time | | RRT | Total | Final pH adjusting |
|---|---|---|---|---|---|---|
| Formulation | temp. | point | | 0.66 | Imp. % | agent concentration |
| Formulation 1, T- | 40° C. | START | | <0.05 | <0.05 | 0.0135 mg/mL |
| AWE001 | | 3 | month | <0.05 | <0.05 | tartaric acid |
| 0.2 mg/ml PEP with | | | | | | |
| tartaric acid. Without | | | | | | |
| nitrogen purging, with | | | | | | |
| overwrap | | | | | | |
| Formulation 2, 077 | 40° C. | START | | <0.05 | 0.1 | 0.0116 mg/mL |
| 0.08 mg/ml PEP with | | 3 | month | <0.05 | 0.1 | Tartaric acid |
| tartaric acid. Without | | | | | | |
| nitrogen purging, with | | | | | | |
| overwrap | | | | | | |
| Comparative | 40° C. | START | | N/A | 0.1 | 0.1345 mg/mL |
| Formulation 4, 025 | | 3 | months | N/A | 0.8 | citric acid |
| 0.2 mg/ml PEP with | | | | | | |
| citric acid. With | | | | | | |
| nitrogen purging, with | | | | | | |
| overwrap | | | | | | |

Formulations 1 and 2 using tartaric acid, even without oxygen removal during manufacture, has reduced total impurities compared to the citric acid formulation with N₂ purging.

### Example 2

Table 2 shows formulations of 0.2 mg/ml Phenylephrine (PEP) with an overwrap. Formulation 1 is produced without any nitrogen purging of the solution during product manufacture and formulation 028 is the same as Formulation 1 but has been produced with nitrogen purging to remove oxygen from solution during compounding. Comparative formulation 4 is with citric acid as the pH adjusting agent and it is produced nitrogen purging to remove oxygen from solution during compounding.

TABLE 2
STABILITY AT 40° C. WITH OVERWRAP

| | | | | | | Final pH |
|---|---|---|---|---|---|---|
| | Condition | Time | | RRT | Total | adjusting agent |
| Formulation | temp. | point | | 0.66 | Imp. % | concentration |
| Formulation 1, T-AWE001 | 40° C. | START | | ND | <0.05 | 0.0135 mg/mL |
| 0.2 mg/ml PEP with tartaric | | 3 | month | <0.05 | <0.05 | tartaric acid |
| acid. | | 6 | month | 0.11 | 0.2 | |
| Without nitrogen purging, | | | | | | |
| with overwrap | | | | | | |
| Formulation 3, 028 | 40° C. | START | | ND | <0.05 | 0.0175 mg/mL |
| 0.2 mg/ml PEP with tartaric | | 3 | month | <0.05 | 0.05 | Tartaric acid |
| acid. | | 6 | month | 0.15 | 0.49 | |
| With nitrogen purging, with | | | | | | |
| overwrap | | | | | | |
| Comparative Formulation 4, | 40° C. | START | | N/A | 0.1 | 0.1345 mg/mL |
| 025 | | 3 | months | N/A | 0.8 | citric acid |
| 0.2 mg/ml PEP with citric | | 6 | months | 0.66 | 2.4 | |
| acid. With nitrogen purging, | | | | | | |
| with overwrap | | | | | | |

As can be seen from Table 2, when tartaric acid is used as the pH-adjusting agent, the level of total impurities is much lower in Formulation 1 compared to Formulation 28. It can also be seen from Table 2, when tartaric acid is used as the pH-adjusting agent, the stability after 6 months is at least about 5-fold improved compared to the Comparative formulation number 4 (i.e., with citric acid and purged with nitrogen).

1. A product comprising a ready-to-administer injectable formulation, the formulation comprising phenylephrine or a pharmaceutically acceptable salt thereof, an osmolality adjusting agent, and a pH adjusting agent that is tartaric acid in an amount less than 0.05 mg/ml, wherein the formulation is contained in a flexible plastic container.

2. A product comprising a ready-to-administer injectable formulation, the formulation comprising phenylephrine or a pharmaceutically acceptable salt thereof, an osmolality adjusting agent and a pH adjusting agent that is tartaric acid in an amount less than 0.05 mg/ml; wherein the formulation is contained in a flexible plastic container; wherein oxygen has not been removed from the product; and wherein the level of total impurities is less than 0.40% after 3 months at 40 degrees Celsius as determined by HPLC.

3. The product according to claim 1, wherein the osmolality adjusting agent is sodium chloride.

4. The product according to claim 3, wherein the concentration of sodium chloride is 8 to 10 mg/ml.

5. The product according to claim 4, wherein the concentration of sodium chloride is 9 mg/ml.

6. The product according to claim 1, wherein the pH of the formulation is 3.7 to 4.4.

7. The product according to claim 6, wherein the pH of the formulation is 4.0.

8. The product according to claim 1 wherein the phenylephrine is phenylephrine hydrochloride.

9. The product according to claim 1, wherein the concentration of phenylephrine as phenylephrine hydrochloride is 0.05 mg/ml to 0.5 mg/ml.

10. The product according to claim 8, wherein the concentration of phenylephrine as phenylephrine hydrochloride is 0.08 mg/ml.

11. The product according to claim 8, wherein the concentration of phenylephrine as phenylephrine hydrochloride is 0.16 mg/ml.

12. The product according to claim 8 wherein the concentration of phenylephrine as phenylephrine hydrochloride is 0.20 mg/ml.

13. The product according to claim 8 wherein the concentration of phenylephrine as phenylephrine hydrochloride is 0.40 mg/ml.

14. The product according to claim 1, wherein the concentration of tartaric acid is 0.001 mg/ml to 0.05 mg/ml.
